(19) United States
(12) Patent Application Publication
(10) Pub. No.: US 2023/0084612 A1
(43) Pub. Date: Mar. 16, 2023

(54) COOLING SYSTEM COMPRISING CELLS FOR RECEIVING A COOLANT

(71) Applicant: VESTEL ELEKTRONIK SANAYI VE TICARET A.S., Manisa (TR)

(72) Inventor: Serdar TOPCUOGLU, Manisa (TR)

(73) Assignee: VESTEL ELEKTRONIK SANAYI VE TICARET A.S., Manisa (TR)

(21) Appl. No.: 17/799,124

(22) PCT Filed: Feb. 14, 2020
(86) PCT No.: PCT/EP2020/053834

§ 371 Date: Aug. 11, 2022

(51) Int. Cl. A61F 7/10; F25D 31/00; A61F 7/02

(52) U.S. Cl. CPC A61F 7/103; F25D 31/007; A61F 7/02; A61F 2007/0056

(57) ABSTRACT

A cooling system having a multitude of fluidly coupled cells for receiving a coolant is disclosed. The cooling system is characterized in that the cooling system has a flexible cover that covers the multitude of cells, an air gap between the flexible cover and the cells and an air pump for modifying the air gap.

### TECHNICAL FIELD

The invention relates to a cooling system comprising cells for receiving a coolant.

Technological Background

A standard refrigerator contains two main parts, namely a cooler and a freezer. In the freezer section a multitude of differently shaped passive, plastic ice making reservoirs can be placed. If the ice making reservoirs are filled with water and placed in the freezer, the water freezes and after some time ice occurs. This ice can be removed from the ice making reservoirs in small portions, e.g. in the form of ice cubes. These ice cubes can be used in drinks, in particular soft drinks or ice coffee or alcohol-free cocktails and juice, with the purpose of cooling the liquid where the ice is placed. The ice might also be used with a medical purpose, e.g. for cooling a wound or bruise.

These ice cubes however, have the disadvantage that they melt when they are in contact with a warm environment. In particular, when such an ice cube is put in any drink, which is warmer than the temperature of the freezing temperature of the water, the ice cube cools the drink but at the same time it melts and dilutes the drink. The same problem occurs in medical applications, where the melt water of the ice cubes might infect any open wounds.

### SUMMARY OF THE INVENTION

It is object of the present invention to provide a cooling system, which does not show the above-mentioned disadvantages.

This object is solved by a cooling system according to the independent claim. Preferred embodiments are given by the dependent claims.

According to the invention, a cooling system is provided, which comprises a multitude of fluidly coupled cells for receiving a coolant. The cooling system is characterized in that it comprises a flexible cover, which covers the multitude of cells. The cooling system further comprises an air gap between the flexible cover and the cells, and an air pump for modifying the air gap. The cooling system will hereinafter also be referred to as system or apparatus.

A cell is formed within a body, and can be filled with media, in particular liquid. For example, such a cell can be a portion of a plastic bag, which forms the cell. The inner space enclosed by the cell will also be referred to as cavity. The body, wherein the cells are formed, can for example be made of plastic. The body is preferably at least partially elastically deformable. Preferably, the cavities are preformed so that they exists even before the cells are filled. The system contains at least two cells and preferably more than two cells. There is no upper limit of number of cells. For example, a cooling system can comprise 10 to 100 cells or 1000 cells and more. The multitude of the cells will also be referred to as the entirety of cells.

A coolant is preferably a liquid that is to be cooled down to or below its freezing temperature. According to an embodiment, the coolant is water. When a frozen coolant is brought into an environment, which has a higher temperature, in particular above the freezing temperature of the coolant, the coolant receives thermal energy from the environment and thus cools the environment. As the frozen coolant receives thermal energy from the environment, the coolant warms up. If the temperature of the frozen coolant increases above its melting temperature, the coolant melts. During such a phase transition of the coolant, the specific heat capacity of the coolant changes. The phase transition is also accompanied with a change of the volume of the coolant. The phase transition can further results in an inhomogeneous heat transport. In particular, within a cell the frozen coolant phase and the liquid coolant phase can be coexistent. In this case, the frozen coolant and the liquid coolant phase simultaneously participate in the heat transport.

The cells can receive coolant, for example, by filling the coolant into the cells during the production process of the cooling system. However, there may be means provided at the system to fill the coolant into the cells after the production of the system. Such means can be a re-closable opening in one of the cells.

The cells are fluidly coupled, that means that the coolant can flow from one cell to another cell. This can for example be realized, if the cells overlap at least partially, so that the cavity of a cell and the cavity of another cell enclose a joint volume. A fluid coupling can also be realized, for example, by using tubes or hollow connectors that connect the cavities of two cells. Typically, if all cells are located in the same environmental conditions, for example atmospheric pressure, and every cell is filled in such a fashion that a fluid coupling is possible, the filling level of the coolant of each cell will equalize, by a flow of coolant between the different cells until every cell is filled to the same amount. This is called the principle of communication vessels.

According to an embodiment, the shape of the cells is ellipsoidal, in particular spherical. The ellipsoidal cells can be elongated in one direction, for example in the direction of the fluid coupling. However, in a preferred embodiment the cells have a spherical shape, as spheres have a very large volume-to-surface ratio. This ensures that coolant in the cells remain in the frozen state for a long time.

The system comprises a flexible cover that covers the multitude of cells. A flexible cover according to the present invention refers to a cover that, for example, can be twisted without breaking or tearing and can withstand distortions using for example tension and shear forces to a small amount without breaking or tearing. The cover covers the multitude of cells. Preferably, the cover encloses the body, wherein the cells are formed. In particular, the cover can wrap around the entirety of cells. The cover can for example be a foil, in particular a flexible foil.

The cover preferably thermally couples the environment to the cells and mediates the heat and energy transfer between the cells and the environment. As the environment does not have direct contact to the cells due to the cover, the cover can be used to regulate the heat transfer between the environment and the cells. For example, metallic covers are very well suited for thermal transport. In contrast, a cover made of aerogel, is very poorly suited for heat transport. The cover therefore preferably is made of material with high a thermally conductivity. In particular, the cover is preferably made of metal or comprise metal for example as a metal coating or metal grid.

The system comprises an air gap between the cells and the cover. The air gap between the cells and the cover can have a continuous height. In some embodiments, the height of the air gap, that means the distance between the outside of the cells and the cover may vary. For example, the air gap can have a greater height at connecting point of two cells or tubes or hollow connectors connecting adjacent cells than at the center of the cell. The air gap can be modified by an air pump. Modifying the air gap means that the height of the air gap is changed in at least a section of the system.

In one embodiment, the cover does not touch the outside of the cells when air is pumped between the cover and the cells. In another embodiment, only the uppermost and lowermost points of the cells touch the cover, when air is pumped between the cover and the cells. In yet another embodiment, one side of cells does not touch the cover, when air is pumped in and the opposite side of the cells does not touch the cover. In all of these cases, the heat transport from the cells to the environment and vice versa is very poor, where air is present between the cells and the cover. In particular, when the cover does not touch the cells, the cells are almost thermally isolated from the environment by the air gap. A consequence is a very low cooling rate of the environment. However, if the air is pumped out of the system, the flexible cover is in direct contact to the cells and thermally couples the cells to the environment. A consequence is a high cooling rate of the environment. In addition, the state where the cover is in direct contact with the cells allows for a rapid cooling of coolant inside the cells.

The air gap can be modified by using the air pump. For example, the air pump is a push-pull pump. However, it is also possible that an electrical pump automatically adjusts the air gap to regulate the heat transport between the cells and the environment. The air pump pumps air into the air gar, or withdraws air from the air gap, depending on the set pumping direction.

For example, the user can put the cooling system, which is filled with a coolant, into a refrigerator to cool the coolant below its freezing temperature. Afterwards, the cooling system can be brought into contact with a body-to-be-cooled. If the cooling system is brought into contact with, for example, a surface of a body, the cooling system can decrease the temperature of the touched surface by about 10 to 15 degrees Celsius and keeps this performance up to two to four hours, depending on the air gap and the coolant.

With the present invention, a number of advantages can be achieved. Firstly, the cavity of cells are provided in a body, the space, wherein the coolant can be received, is a closed space. Thereby, even if a frozen coolant melts during usage of the system, the melted coolant is contained within the system and cannot leak. Thereby, dripping of the coolant can be avoided and for example, infections during medical use of the system for wounds can be avoided. Secondly, by providing a flexible cover for the multitude of cells and a pump for modifying the air gap between the cells and the cover the amount of heat received by the coolant or cooling function of the coolant can be adapted to the current needs.

According to one embodiment, the cells are fluidly coupled in a matrix-like structure.

A matrix-like structure, which is also referred to as a matrix structure hereinafter, is a one- or two-dimensional structure, where the cells are located in a certain finite distance to each other. For example, a simple chain of cells, where all cells have the same distance to their neighbors is a one-dimensional matrix-like structure. For example, when the cells are placed in a grid, where the distance of all cells to their nearest neighbors is the same, that means where the grid is a regular grid, the cells build a two dimensional matrix-like structure. In particular, the cells could then be coupled in parallel and in series. However, the cells can also be placed on a two dimensional irregular grid, such as the Penrose grid. The cells in the grid can have the same volume; however, it is possible, that some cells have a different volume.

By providing the cells in a matrix-like structure and preferably in a two-dimensional matrix structure, the surface via which the system can cool an object can be maximized. In addition, in particular in a two-dimensional structure, the matrix structure allows for an exchange of coolant between a large number of cells.

According to a preferred embodiment, a cell comprises an inlet. The inlet serves for providing coolant into the cells of the system. By providing an inlet, the user of the cooling system can supply the coolant to the system and can also exchange the coolant, when needed. Preferably, the system comprises an inlet cover. The inlet cover may be provided as an internal element such as a valve. For example, when the cells are fully filled with the coolant and the matrix-like structure is rotated up side down, an inlet cover can automatically close the inlet. In an alternative embodiment, the inlet cover can be an external element such as a cap. The inlet cover locks the system against water leakage.

According to one embodiment, at least one of the cells is a volume-change-tolerant-cell.

Preferably, all cells of the system are volume-change-tolerant cells. A volume-change-tolerant cell, allows the coolant to expand and contract its volume during a phase transition. This can, for example, be achieved by cells, which are made of flexible plastic. In this case, the expansion of the coolant during the phase transition cannot harm the cell. In addition, the volume-change-tolerant cell can be used as the first cell to be filled in the system. In that case, the inlet of the system is provided at the volume-change-tolerant-cell. If the cells are for example filled via a water tap, the property of volume-change-tolerance of the first cell can be advantageous to withstand the larger amount of water and high water pressure from the tap received in the first cell.

According one embodiment, the cells are fluidly coupled in a two-dimensional matrix like structure and the volume-change-tolerable cell is located in a corner of the matrix-like structure.

For example, when the multitude of cells has a rectangular shape, the column-change-tolerable cell can be located at a corner of the rectangle. By using a volume-change-tolerant cell at the corner of a matrix-like structure, this cell can be used as the first cell and the entire matrix can be filled easily.

According to a preferred embodiment, the cooling system comprises a coolant pump for pumping the coolant through at least one of the cells of the multitude of cells. Preferably, the coolant pump serves for pumping the coolant through at least to and further preferably through all cells of the system example, the coolant pump can pump the liquid portion of the coolant from one cell to another cell. In particular, the coolant pump can circulate the liquid portion of the coolants through the matrix-like structure.

The matrix-like structure of cells might be exposed to different environmental temperatures. For example, a local heat exposure might effect some of cells, where the coolant is frozen, of the matrix-like structure. The frozen coolant of the cells, where local heat exposure takes place, might melt quicker than frozen coolant in other cells. In an extreme case, during such local heat exposures, the coolant in a cell might melt completely and the liquid coolant heats up well above its freezing temperature until it has the same temperature as the local heat source, that means thermalizes. In this situation, the liquid coolant in the cell cannot receive any further thermal energy from the local heat source. A circulation of the liquid coolant through the other cells can now result in cooling down the liquid coolant. When the cooled down coolant is circulated to the local heat exposure again, it can receive further thermal energy and cool down the local heat source. Hence, by using a coolant pump, the cooling process of a local heat source involves the multitude of cells and thus the summed-up or combined heat capacity of the coolant of the multitude of cells.

The provision of a coolant pump is also advantageous in view of the fact that outer cells of the systems are normally in contact with about half of their surface with the cover. During high cooling performance, the system's outer cells melt first, due to the cover that wraps around about half of the outer cell circumference. Thus, the cover interacts more with the outer cells than with the inner cells. Hence, a water frame occurs in the circumference of the system. The coolant pump can compensate these local heating effects by cooling the liquid coolant in the outer cells, by circulating the liquid coolant along frozen cells, in particular inner cells.

According to one embodiment, the coolant pump comprises a battery that can be charged externally. Preferably, the battery can be charged via a cable. In this embodiment, the cable is attached to the system. A charging unit for the battery, for example, may be provided in a refrigerator. For example, a DC charger circuit can be added to the main board of refrigerator and the cooling system can then be charged via the freezer compartment of the refrigerator. However, it is also possible, that the cooling system can be charged via a USB port, in particular using a power bank.

The coolant pump preferably has a low energy consumption, in particular an energy consumption between 0.3 W to 0.9 W. The pump can also work with a low torque. This enables a long usage dura-tion with the available battery capacity.

According to one embodiment, the coolant pump is located at an opposite end of the matrix-like structure to the inlet of the structure. Preferably, if the inlet is positioned at a corner of a two-dimensional matrix-like structure, the coolant pump is provided at a cell in the opposite corner of the matrix.

The opposite corner is in the case of a rectangular matrix-like structure, the diagonally opposite corner. By providing the coolant pump at the opposite corner to the inlet, a reliable mixing of the coolant within the structure can be ensured.

According to a further embodiment of the invention, the inlet is lockable, where in the locked state the cell, where the inlet is located, closes a circulation path through at least two of the multitude of cells. In one example, the cell, where the inlet is provided comprises a rotatable tubular mechanism that allows the coolant in one rotation state to flow through the inlet into the cells, whereas in another rotation state the coolant is only able to flow into other cells, but not back through the inlet.

However, the cooling system can also comprise a rotatable inlet cover that can be attached, for example screwed, onto the inlet. If the inlet cover is rotated further, when the inlet is already closed, an internal rotatable inlet mechanism can rotate with the inlet cover and open a passage to an adjacent cell to enable a circulation of the coolant.

According to one embodiment, the air pump is a manual push-pull pump.

For example, if the user wants a lower cooling performance, he manually pushes air into the system using the push-pull pump, that means that the pump is compressed. With air being provided by the pump to the air gap, the height of the air gap increases between the cells and the cover, which decreases the thermal conductivity. If the user wants a higher cooling performance, the user removes the compression force on the manual air push-pull pump and some of the air escapes the cooling system. The air gap decreases and increases the total thermal conductivity from the cells to the environment. The systems cooling performance can thus be set by user manually.

According to a further embodiment, the flexible cover comprises copper, aluminum and/or silver.

Metals are very well suited for heat transport application, and can thus be used to transport heat to frozen coolant in cells and to cool coolant in the cells. The cover can be a metal foil. Alternatively, the cover can comprise a metal coating or a metal grid included in the base material of the cover, which may be plastic.

### BRIEF DESCRIPTION OF THE DRAWINGS

The present disclosure will be more readily appreciated by reference to the following detailed description when being considered in connection with the accompanying drawings in which:

FIG. 1 is a schematic view of one embodiment of the cooling device,

FIG. 2A-2D are cross sectional views along line L1 of FIG. 1, and

FIG. 3A, B, C are schematic views of different applications of the cooling system.

### DETAILED DESCRIPTION OF PREFERRED EMBODIMENTS

In the following, the invention will be explained in more detail with reference to the accompanying figures. In the Figures, like elements are denoted by identical reference numerals and repeated description thereof may be omitted in order to avoid redundancies.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

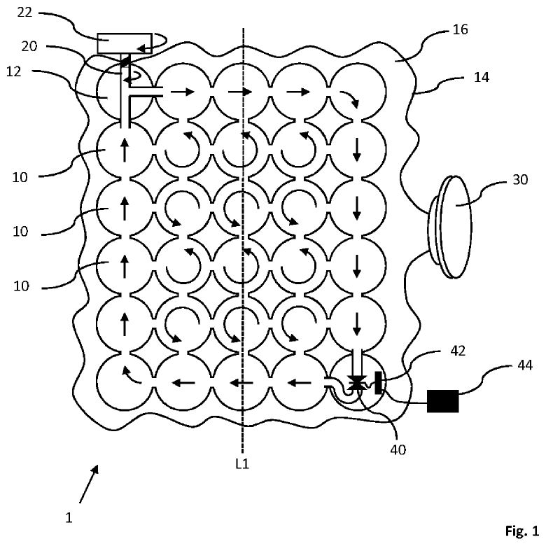

FIG. 1 shows a schematic view of an embodiment of the cooling system 1 according to the present invention. The cooling system 1 comprising a multitude of fluidly coupled cells 10 for receiving a coolant. The cooling system 10 further comprises a flexible cover 14, which covers the multitude of cells 10. In the depicted embodiment, the cooling system 1 has thirty spherical cells 10. The cells 10 are positioned in a regular grid, that means that the distance to the nearest neighbor of every cell 10 is constant throughout the matrix-like structure. The cooling system 1 comprises an inlet 20 for providing coolant (not shown) to the cells 10. The inlet 20 is closed with an inlet cover 22, which in the depicted embodiment is a cap, which can be screwed onto the inlet 20. The cell 10 at which the inlet 20 is provided in the depicted embodiment is a volume-change-tolerant cell 12. The inlet 20 is provided at a cell 10, which is positioned at one corner of the matrix-like structure. The cooling system 1 further comprises an air pump 30. The air pump 30 is connected to the cover 14 for introducing air into an air gap between the cells 10 and the cover 14. Finally, the cooling system 1 in the depicted embodiment has a coolant pump 40. The coolant pump 40 is provided in a cell 10 at the opposite corner of the matrix-like structure from the corner where the inlet 20 is provided. The cooling system 1 has a battery 42, which supplies the coolant pump 40. In FIG. 1, the cooling system 1 is shown in a state where the battery 42 is connected to a battery charger 44, which is preferably external to the cooling system 1 and can for example be provided in a refrigerator (not shown).

In the view of FIG. 1, the upper left cell 10 is the volume-change-tolerant cell 12, which also contains the coolant inlet 20 with a coolant inlet cover 22. The user can open the coolant inlet cover 20 and fill the cells 10 with water as a coolant. For example, the user can fill the cells 10 up to approximately 85%, such that the expansion of the water during the freezing phase transition does not harm the cells 10. Afterwards, the water inlet cover 22 seals the inlet 20 by screwing the inlet cover 22 on the inlet 20. During this operation, the horizontal pipe of the water inlet 20, which is connected to a vertical pipe of the water inlet 20, rotates with the water inlet cover 22 and opens the first horizontal passage to the adjacent cell 10. This enables the water to flow to all adjacent cells, which is a prerequisite for a circulation of the water in the cooling system 1.

After the filling process of the cells 10, the user places the cooling system 1 into a refrigerator. In particular, the cooling system 1 can be cooled in all refrigerator types. For example, the user can connect the refrigerator main board to the battery charger 44, which can charge the battery 42 of the coolant pump 40, while the water freezes. After the water turned into ice, the user can take the cooling system 1 and bring it into a warmer environment, where the frozen coolant then cools the environment.

The multitude of cells 10 is surrounded by a flexible cover 14, which may be made of a flexible copper-aluminum-silver foil. This cover 14 is connected to the manual air push-pull pump 30 for providing air between the cells 10 and the cover 14, which determines the thermal conductivity and the cooling performance of the cooling system 1. The user can modify the extension of the air gap 16 using the manual air push-pull-pump 30.

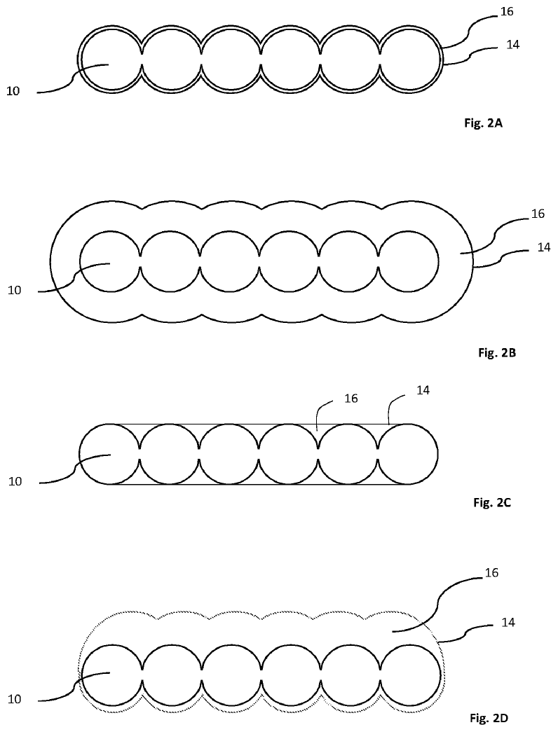

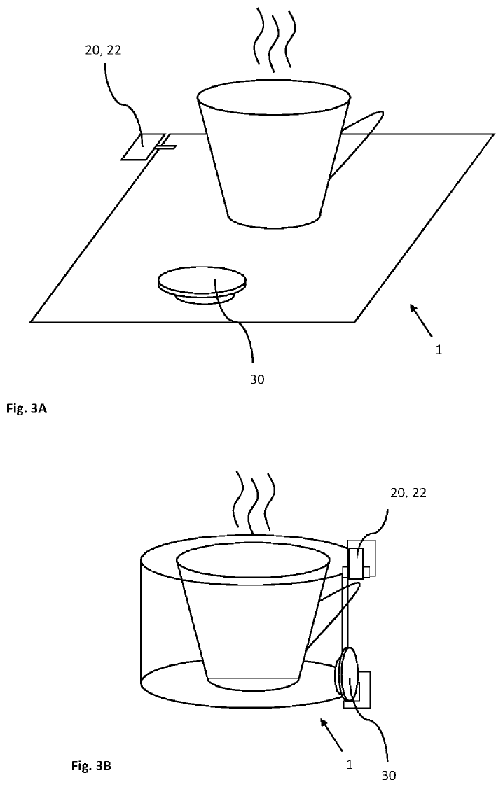

FIG. 2 shows a cross sectional view along line L1 in FIG. 1. In FIG. 2A the state of the flexible cover 14 is shown, when the extension of the air gap 16 is minimized using the air pump 30. The flexible cover 16 is in direct contact with the cells 10 and thus enables a high performance cooling.

In FIG. 2B the state of the flexible cover 14 is shown, when the extension of the air gap 16 is maximized using the air pump 30. Between the flexible cover 14 and the cells 10 a large air gap 16 occurs, which isolates the cells 10 from the environment, as the flexible cover 14 cannot transport the heat directly to the cells 10.

In FIG. 2C an alternative embodiment of the cooling system 1 in the expanded state of the cover 14 is shown. In this embodiment, the cover 14 is adhered to the top and bottom of the cells 10.

Therefore, the air gap 16 between the cover 14 and the cells 10 is only increased in the areas of the transition between two adjacent cells 10.

In FIG. 2D another embodiment of the expanded state of the cover 14 is shown. In this embodiment, the air gap 16 is only increased on one side of the cells 10 whereas the cover 14 is in contact with the cells 10 on the other side of the cells 10, in FIG. 2D the lower side. The cover 14 can be adhered to the lower side of the cells 10 or the volume or the delivery of air from the air pump 30 can be blocked from entering the gap between the bottom side of the cells and the cover 14. This embodiment can be advantageous, since the side with the cover 14 in contact with the cells 10 can be used to cool an object, while the upper side with the increased air gap 16 maintains the thermal insulation of the cells 10.

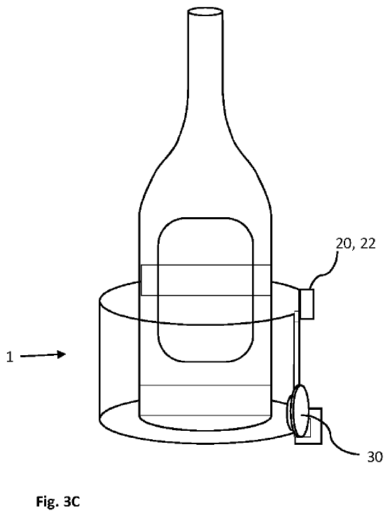

FIG. 3A shows the cooling system 1 during a cool down process of a cup filled with a hot beverage. The frozen coolant, in particular water, in the cells 10 underneath the cup will melt quickly in comparison to frozen coolant in adjacent cells. The melt water can then be circulated using the coolant pump 40 though the cells 10, where the liquid water cools down and is able to transport the heat away from the bottom of the cup. The coolant pump 40 receives its electrical energy from the battery 42.

FIG. 3B shows an alternative usage of the cooling system 1, where the cooling system 1 is wrapped around the cup that is filled with a hot beverage. In this configuration, the contact area of the cooling system 1 is larger than in FIG. 3A, which results in a faster cool down of the beverage. The place, where the ends of the rectangular cooling system 1 meet, can be attached to each other using for example a magnetic fixation.

In both FIGS. 3A and 3B, the object to be cooled and thus the environment is very hot. If a fast cool down is required, the air can be pumped out of the air gap 16 using the air pump 30. In this high performance setting, the outer cup surface touches the flexible cover 14, whereas the other side of the flexible cover 14 touches the cells 10. Hence, the flexible cover 14 mediates the heat transfer from the hot cup the frozen coolant in the cells 10.

FIG. 3C shows an alternative usage of the cooling system 1, where the cooling system 1 is wrapped around a precooled bottle of wine. As the wine is precooled, it is not necessary to use the high performance setting of the cooling system 1, that means that air can be pumped into the air gap 16. Hence, the thermal insolation between the cells 10 and environment increases, which prevents the frozen coolant in cells 10 from melting quickly. This makes it possible to keep the temperature of the precooled bottle for a long time.

### LIST OF REFERENCE NUMERALS

1 Cooling system
10 Cell
12 Volume-change-tolerant cell
14 Flexible cover
16 Air gap
20 Inlet
22 Inlet cover
30 Air pump
40 Coolant pump
42 Battery
44 Battery charger

1. A cooling system comprising a multitude of fluidly coupled cells (10) for receiving a coolant, characterized in that the cooling system (1) comprises a flexible cover (14), that covers the multitude of cells (10), an air gap (16) between the flexible cover (14) and the cells (10) and an air pump (30) for modifying the air gap (16).

2. The cooling system claim 1, characterized in that the cells (10) are fluidly coupled in a matrix-like structure.

3. The cooling system of claim 1, characterized in that a cell comprises an inlet (20) for providing coolant into the cells (10).

4. The cooling system of claim 1, characterized in that at least one of the cells (10) is a volume-change-tolerant-cell (12).

5. The cooling system of claim 4, characterized in that the cells (10) are fluidly coupled in a two-dimensional matrix-like structure and the volume-change-tolerable cell (12) is located in a corner of the matrix-like structure.

6. The cooling system of claim 1, characterized in that the cooling system (1) comprises a coolant pump (40) for pumping the coolant through at least one of the cells (10).

7. The cooling system of claim 6, characterized in that the coolant pump (40) comprises a battery (42) that can be charged externally.

8. The cooling system of claim 6, characterized in that the coolant pump (40) is located at an opposite end of the matrix-like structure to the inlet (20).

9. The cooling system of claim 3, characterized in that the inlet (20) is lockable, wherein in the locked state the cell (10), where the inlet (20) is located, closes a circulation path through at least two of the multitude of cells (10).

10. The cooling system of claim 1, characterized in that the air pump (30) is a manual push-pull pump.

11. The cooling system of claim 1, characterized in that the flexible cover (14) comprises copper, aluminum and/or silver.

12. The cooling system of claim 1, characterized in that the coolant is water.

13. The cooling system of claim 1, characterized in that the shape of the cells (10) is ellipsoidal, in particular spherical.
